(19) United States
(12) Patent Application Publication
(10) Pub. No.: US 2024/0169029 A1
(43) Pub. Date: May 23, 2024

(54) Feature calculation apparatus, feature calculation method, and storage medium

(71) Applicant: NEC Corporation, Tokyo (JP)

(72) Inventors: Keisuke SUZUKI, Tokyo (JP); Riki Eto, Tokyo (JP)

(73) Assignee: NEC Corporation, Tokyo (JP)

(21) Appl. No.: 18/385,195

(22) Filed: Oct. 30, 2023

(30) Foreign Application Priority Data
Nov. 15, 2022 (JP) 2022-182867

(51) Int. Cl. G06F 18/2135; G01N 33/00

(52) U.S. Cl. CPC G06F 18/2135; G01N 33/0001

(57) ABSTRACT

To provide a technique of calculating an inverse Laplace transform as a feature of a signal y(t) which is represented as a combination of a Laplace transform of a discrete measure and an offset b, a feature calculation apparatus includes a calculation section that calculates, for each of n consecutive intervals (α₀, α₁], (α₁, α₂], . . . , (αₙ₋₁, αₙ], a value ν_{y-b}((αₖ₋₁, αₖ]) as a feature of a signal y(t) approximated by formula (1) below and including an offset b, the value ν_{y-b}((αₖ₋₁, αₖ]) being approximated by formula (2) below:

$$y ( t ) = b + {\sum }_{k = 1}^{K} {ξ}_{k} {e}^{- {β}_{k} t} \qquad ( 1 )$$

$${v}_{y - b} ( ( {α}_{k - 1} , {α}_{k} ] ) = {\sum }_{k : {α}_{k - 1} < k \leq {α}_{k}} {ξ}_{k} . \qquad ( 2 )$$

### CROSS REFERENCE TO RELATED APPLICATIONS

This application is based upon and claims the benefit of priority from Japanese Patent Application No. 2022-182867 filed on Nov. 15, 2022, the disclosure of which is incorporated herein in its entirety by reference.

### TECHNICAL FIELD

The present invention relates to a technology for calculating a feature of a signal.

### BACKGROUND ART

The technique of using the inverse Laplace transform to determine an original function has conventionally been in use. For example, Non-Patent Literature 1 discloses the technique of using the inverse Laplace transform to determine an original continuous measure. Non-Patent Literature 2 discloses the technique of formally treating a discrete measure as a continuous measure with use of a δ function and inverse-transforming a result of a Laplace transform of the continuous measure. Non-Patent Literature 2 indicates formally treating a discrete measure as a continuous measure with use of a linear sum of a δ function and inverse-transforming a result of a Laplace transform of the continuous measure.

### CITATION LIST

### Non-Patent Literature

[Non-Patent Literature 1]
H. Fujiwara et. al., “Real inversion of the Laplace transform in numerical singular”, Journal of Analysis and Applications, January 2008 value decomposition
[Non-Patent Literature 2]
Donald G. Gardner, et. al., “Method for the Analysis of Multicomponent Exponential Decay Curves”, Mar. 17, 1959

### SUMMARY OF INVENTION

### Technical Problem

However, the technique disclosed in Non-Patent Literature 1 is a method that uses manipulation in a functional space, and it is not obvious whether or not the technique is applicable to a discrete measure which has no density function with respect to a Lebesgue measure. With the technique disclosed in Non-Patent Literature 2, it is not even possible to determine g in a case where g is a generalized function. It is thus not possible to determine a discrete measure either.

An example aspect of the present invention has been made in view of the above problems, and an example object thereof is to provide a technique that makes it possible to calculate an inverse Laplace transform as a feature of a signal y(t) represented as a combination of a Laplace transform of a discrete measure and an offset b.

### Solution to Problem

A feature calculation apparatus in accordance with an example aspect of the present invention includes at least one processor, the at least one processor carrying out a calculation process of calculating, for each of n consecutive intervals (α₀, α₁], (α₁, α₂], . . . , (αₙ₋₁, αₙ], a value ν_{y-b}((αₖ₋₁, αₖ]) as a feature of a signal y(t) approximated by formula (1) below and including an offset b, the value ν_{y-b}((αₖ₋₁, αₖ]) being approximated by formula (2) below.

A feature calculation method in accordance with an example aspect of the present invention includes calculating, by at least one processor and for each of n consecutive intervals (α₀, α₁], (α₁, α₂], . . . , (αₙ₋₁, αₙ], a value ν_{y-b}((αₖ₋₁, αₖ]) as a feature of a signal y(t) approximated by formula (1) below and including an offset b, the value ν_{y-b}((αₖ₋₁, αₖ]) being approximated by formula (2) below.

A computer-readable non-transitory storage medium in accordance with an example aspect of the present invention stores therein a program for causing a computer to carry out a process of calculating, for each of n consecutive intervals (α₀, α₁], (α₁, α₂], . . . , (αₙ₋₁, αₙ], a value ν_{y-b}((αₖ₋₁, αₖ]) as a feature of a signal y(t) approximated by formula (1) below and including an offset b, the value ν_{y-b}((αₖ₋₁, αₖ]) being approximated by formula (2) below.

$$y ( t ) = b + {\sum }_{k = 1}^{K} {ξ}_{k} {e}^{- {β}_{k} t} \qquad ( 1 )$$

$${v}_{y - b} ( ( {α}_{k - 1} , {α}_{k} ] ) = {\sum }_{k : {α}_{k - 1} < k \leq {α}_{k}} {ξ}_{k} \qquad ( 2 )$$

where K and n are each a natural number, b and ξ₁, ξ₂, . . . , ξ_{K} are each a real number, β₁, β₂, . . . , β_{K} are each a positive number, and α₀, α₁, . . . , αₙ are each a positive number that satisfies βₘᵢₙ=α₀<α₁< . . . <αₙ=βₘₐₓ where βₘᵢₙ≤min{β₁, β₂, . . . , β_{K}} and βₘₐₓ≥max{β₁, β₂, . . . , β_{K}}.

### Advantageous Effects of Invention

According to an example aspect of the present invention, it is possible to calculate an inverse Laplace transform as a feature of a signal y(t) represented as a combination of a Laplace transform of a discrete measure and an offset b.

### BRIEF DESCRIPTION OF DRAWINGS

FIG. 1 is block diagram illustrating a configuration of a feature calculation apparatus in accordance with a first example embodiment.

FIG. 2 is a flowchart illustrating a flow of a feature calculation method in accordance with the first example embodiment.

FIG. 3 is block diagram illustrating a configuration of a feature calculation apparatus in accordance with a second example embodiment.

FIG. 4 is block diagram illustrating a configuration of a feature calculation apparatus in accordance with a third example embodiment.

FIG. 5 is a block diagram illustrating a configuration of a smell determination apparatus in accordance with a fourth example embodiment.

FIG. 6 is a flowchart illustrating a flow of a smell determination method in accordance with the fourth example embodiment.

FIG. 7 is a diagram illustrating an example of display of a result of determination on a smell.

FIG. 8 is a diagram illustrating an example of a signal y(t) measured by an MSS sensor.

FIG. 9 is a diagram illustrating an example of a signal y(t) represented with use of a feature calculated by the smell determination apparatus in accordance with the fourth example embodiment.

FIG. 10 is a diagram illustrating an example of a signal y(t) represented with use of a feature calculated by the smell determination apparatus in accordance with the fourth example embodiment.

FIG. 11 is a flowchart illustrating a flow of a feature calculation method in accordance with a variation of the second example embodiment.

FIG. 12 is a block diagram illustrating a configuration of a computer that functions as an information processing device in accordance with each example embodiment.

### EXAMPLE EMBODIMENTS

### First Example Embodiment

The following will discuss in detail a first example embodiment of the present invention, with reference to drawings. The present example embodiment is a basic form of example embodiments described later.

### (Configuration of Feature Calculation Apparatus)

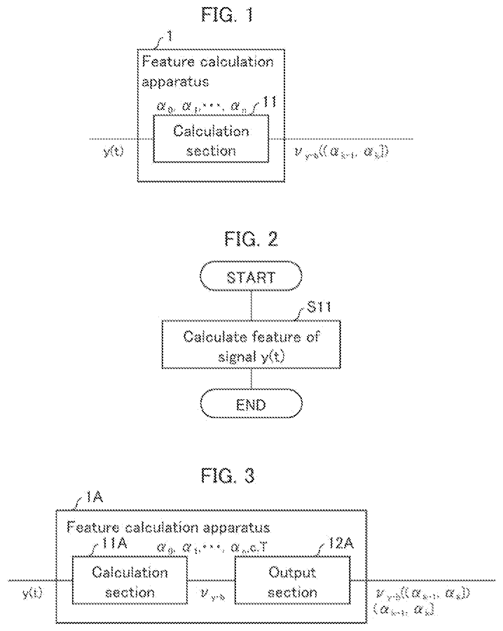

The following will discuss a configuration of a feature calculation apparatus 1 in accordance with the present example embodiment, with reference to FIG. 1. FIG. 1 is a block diagram illustrating a configuration of the feature calculation apparatus 1. The feature calculate apparatus 1 includes a calculation section 11 (calculation means).

The calculation section 11 calculates, for each of n consecutive intervals (α₀, α₁], (α₁, α₂], . . . , (αₙ₋₁, αₙ], a value ν_{y-b}((αₖ₋₁, αₖ]) as a feature of a signal y(t) approximated by formula (11) below and including an offset b, the value ν_{y-b}((αₖ₋₁, αₖ]) being approximated by formula (12) below:

$$y ( t ) = b + {\sum }_{k = 1}^{K} {ξ}_{k} {e}^{- {β}_{k} t} \qquad ( 11 )$$

$${v}_{y - b} ( ( {α}_{k - 1} , {α}_{k} ] ) = {\sum }_{k : {α}_{k - 1} < k \leq {α}_{k}} {ξ}_{k} \qquad ( 12 )$$

where K and n are each a natural number, b and ξ₁, ξ₂, . . . , ξ_{K} are each a real number, β₁, β₂, . . . , β_{K} are each a positive number, and α₀, α₁, . . . , αₙ are each a positive number that satisfies βₘᵢₙ=α₀<α₁< . . . <αₙ=βₘₐₓ where βₘᵢₙ≤min{β₁, β₂, . . . , β_{K}} and βₘₐₓ≥max{β₁, β₂, . . . , β_{K}}.

βₘᵢₙ and βₘₐₓ can be, for example, values inputted by a user of the feature calculation apparatus 1 with use of an input apparatus, or values preset in the feature calculation apparatus 1. α₀, α₁, . . . , αₙ are values by which divided intervals of [βₘᵢₙ, βₘₐₓ] are determined. When βₘᵢₙ and βₘₐₓ are given, α₀, α₁, . . . , αₙ can be set as appropriate within a range of βₘᵢₙ to βₘₐₓ. For example, the calculation section 11 can calculate α₁, . . . , αₙ that equally divide the [βₘᵢₙ, βₘₐₓ] interval. Alternatively, for example, a user can set α₁, . . . , αₙ within the range of βₘᵢₙ to βₘₐₓ. The offset b can be, for example, a value inputted by a user of the feature calculation apparatus 1 with use of an input apparatus, or a value preset in the feature calculation apparatus 1. Alternatively, the feature calculation apparatus 1 can carry out a process of calculating the offset b.

As described above, in the feature calculation apparatus 1 in accordance with the present example embodiment, a configuration is employed in which, for each of the n consecutive intervals (α₀, α₁], (α₁, α₂], . . . , αₙ₋₁, αₙ], the value ν_{y-b}((αₖ₋₁, αₖ]) approximated by formula (12) above is calculated as a feature of the signal y(t) approximated by formula (11) above and including the offset b.

As shown in formula (12) above, ν_{y-b}((αₖ₋₁, αₖ]) calculated by the calculation section 11 corresponds to ξₖ included in formula (11) above representing the signal y(t). As such, by determining ν_{y-b}((αₖ₋₁, αₖ]), it is possible to calculate an inverse Laplace transform as a feature of the signal y(t) which is represented as a combination of a Laplace transform of a discrete measure and the offset b.

### (Feature Calculation Program)

The above-described functions of the feature calculation apparatus can also be realized by a program. A feature calculation program in accordance with the present example embodiment causes a computer to carry out a process of calculating, for each of n consecutive intervals (α₀, α₁], (α₁, α₂], . . . , (αₙ₋₁, αₙ], a value ν_{y-b}((αₖ₋₁, αₖ]) as a feature of a signal y(t) approximated by formula (11) above and including an offset b, the value ν_{y-b}((αₖ₋₁, αₖ]) being approximated by formula (12) above, where K and n are each a natural number, b and ξ₁, ξ₂, . . . , ξ_{K} are each a real number, β₁, β₂, . . . , β_{K} are each a positive number, and α₀, α₁, . . . , αₙ are each a positive number that satisfies βₘᵢₙ=α₀<α₁< . . . <αₙ=βₘₐₓ where βₘᵢₙ≤min{β₁, β₂, . . . , β_{K}} and βₘₐₓ≥max{β₁, β₂, . . . , β_{K}}. The feature calculation program provides the effect of making it possible to calculate an inverse Laplace transform as a feature of the signal y(t) which is represented as a combination of a Laplace transform of a discrete measure and the offset b.

### (Flow of Feature Calculation Method)

The following will discuss a flow of a feature calculation method S1 in accordance with the present example embodiment, with reference to FIG. 2. FIG. 2 is a flowchart illustrating a flow of the feature calculation method S1. Note that steps of the feature calculation method may be carried out by a processor included in the feature calculation apparatus 1 or by a processor included in another system.

At S11, at least one processor calculates, for each of n consecutive intervals (α₀, α₁], (α₁, α₂], . . . , (αₙ₋₁, αₙ], a value ν_{y-b}((αₖ₋₁, αₖ]) as a feature of a signal y(t) approximated by formula (11) above and including an offset b, the value ν_{y-b}((αₖ₋₁, αₖ]) being approximated by formula (12) above, where K and n are each a natural number, b and ξ₁, ξ₂, . . . , ξ_{K} are each a real number, β₁, β₂, . . . , β_{K} are each a positive number, and α₀, α₁, . . . , αₙ are each a positive number that satisfies βₘᵢₙ=α₀<α₁< . . . <αₙ=βₘₐₓ where βₘᵢₙ≤min{β₁, β₂, . . . , β_{K}} and βₘₐₓ≥max{β₁, β₂, . . . , β_{K}}.

As described above, in the feature calculation method S1 in accordance with the present example embodiment, a configuration is employed in which at least one processor calculates, for each of the n consecutive intervals (α₀, α₁], (α₁, α₂], . . . , (αₙ₋₁, αₙ], the value ν_{y-b}((αₖ₋₁, αₖ]) as a feature of the signal y(t) approximated by formula (11) above and including the offset b, the value ν_{y-b}((αₖ₋₁, αₖ]) being approximated by formula (12) above. As such, the feature calculation method S1 in accordance with the present example embodiment provides the effect of making it possible to calculate an inverse Laplace transform as a feature of the signal y(t) which is represented as a combination of a Laplace transform of a discrete measure and the offset b.

### Second Example Embodiment

### (Overview of Feature Calculation Apparatus)

A feature calculation apparatus 1A in accordance with a second example embodiment of the present invention is an apparatus that calculates a feature of a signal y(t). Note here that the signal y(t) is a signal that indicates a result of observation of a subject of analysis. For example, the signal y(t) is a signal outputted from a smell sensor, a signal indicative of a measured value of blood pressure, or an image signal of a diffusion weighted image (DWI). Note that the signal y(t) is not limited to the examples above, and can be a signal pertaining to other matters. For example, the signal y(t) can be a signal indicative of a speed of a P-wave or an S-wave of an earthquake.

In the present example embodiment, formula (21) below is used as a mathematical model of the signal y(t):

$$y ( t ) = b + {\sum }_{k = 1}^{K} {ξ}_{k} {e}^{- {β}_{k} t} \qquad ( 21 )$$

where K is a natural number, b and ξ₁, ξ₂, . . . , ξ_{K} are each a real number, and β₁, β₂, . . . , β_{K} are each a positive number. In the present example embodiment, the offset b is known.

In formula (21) above, b, ξ_{K}, and β_{K} are each a number representing the signal y(t). It can be said that b, ξ_{K}, and β_{K} are features of the signal y(t). By determining b, ξ_{K}, and β_{K}, it is possible to represent the signal y(t) with use of formula (21).

In the present example embodiment, a formula derived from a Levy inversion formula, not a Fourier inversion formula, is used as a technique for reconstructing a discrete measure. By applying a formula derived from a Levy inversion formula instead of a Fourier inversion formula, it is possible to achieve a form that makes it possible to carry out limit manipulation in which an effect of approximation by a continuous measure is cancelled.

### (Configuration of Feature Calculation Apparatus)

FIG. 3 is a block diagram illustrating a configuration of the feature calculation apparatus 1A in accordance with the present example embodiment. The feature calculation apparatus 1A includes a calculation section 11A and an output section 12A. The calculation section 11A calculates, for each of n consecutive intervals (α₀, α₁], (α₁, α₂], . . . , (αₙ₋₁, αₙ], a value ν_{y-b}((αₖ₋₁, αₖ]) as a feature of the signal y(t). Note that n is a natural number, and α₀, α₁, . . . , αₙ are each a positive number that satisfies βₘᵢₙ=α₀<α₁< . . . <αₙ=βₘₐₓ where βₘᵢₙ≤min{β₁, β₂, . . . , β_{K}} and βₘₐₓ≥max{β₁, β₂, . . . , β_{K}}. The method of calculating ν_{y-b}((αₖ₋₁, αₖ]) carried out by the calculation section 11A will be described later.

The calculation section 11A can, for example, obtain the signal which is y(t) inputted through an input/output IF (not illustrated) of the feature calculation apparatus 1A or obtain the signal y(t) which is received through a communication IF (not illustrated) of the feature calculation apparatus 1A. Alternatively, the calculation section 11A can obtain the signal y(t) by reading out the signal y(t) from a storage apparatus built in the feature calculation apparatus 1A or from an external storage apparatus.

βₘᵢₙ and βₘₐₓ used by the calculation section 11A can be, for example, values inputted by a user with use of an input apparatus or values preset in the feature calculation apparatus 1A. α₀, α₁, . . . , αₙ are values by which division of the [βₘᵢₙ, βₘₐₓ] interval is determined. When βₘᵢₙ and βₘₐₓ are given, α₀, α₁, . . . , αₙ can be set as appropriate within a range of βₘᵢₙ to βₘₐₓ. For example, the calculation section 11A can calculate α₁, . . . , αₙ that equally divide the [βₘᵢₙ, βₘₐₓ] interval. Alternatively, for example, a user can set α₁, . . . , αₙ within the range of βₘᵢₙ to βₘₐₓ. The offset b used by the calculation section 11A can be, for example, a value inputted by a user of the feature calculation apparatus 1A with use of an input apparatus, or a value preset in the feature calculation apparatus 1A.

The output section 12A outputs, as a feature of the signal y(t), information indicative of the value ν_{y-b}((αₖ₋₁, αₖ]) and outputs, as additional information, information indicative of an interval (αₖ₋₁, αₖ].

### (Calculation Process Carried Out by Calculation Section)

The calculation section 11A calculates the value ν_{y-b}((αₖ₋₁, αₖ]) as a feature of the signal y(t), with use of formula (23) below and for each of the n consecutive intervals (α₀, α₁], (α₁, α₂], . . . , (αₙ₋₁, αₙ].

$${v}_{y - b} ( ( {α}_{k - 1} , {α}_{k} ] ) = 2 {α}_{k - 1} \times Im [ {\int }_{0}^{c} ( \frac{1}{Γ ( 1 + i ξ )} {\int }_{0}^{T} \{ y ( t ) - b \} {e}^{i ξ logt} dt ) \frac{{e}^{i {ξ log α}_{k - 1}} - {e}^{i ξ log {α}_{k}}}{- 2 πξ} d ξ ] \qquad ( 23 )$$

where Γ(⋅) is a complex gamma function, Im(⋅) is an imaginary part of ⋅, log is a natural logarithmic function, π is a ratio of a circumference of a circle to a diameter thereof, i is an imaginary unit, c is a positive number, and T is a positive number representing measurement time at which the signal y(t) is measured.

As the measurement time T and a range c of integration, values preset in the feature calculation apparatus 1A may be used, or values of the measurement time T and the range c of integration may be inputted by a user with use of an input apparatus and then used by the calculation section 11A. Alternatively, the feature calculation apparatus 1A may calculate values of the measurement time T and the range c of integration.

In the right side of formula (23),

$${\int }_{0}^{c} ( \frac{1}{Γ ( 1 + i ξ )} {\int }_{0}^{T} \{ y ( t ) - b \} {e}^{i ξ logt} dt ) \frac{{e}^{i {ξ log α}_{k - 1}} - {e}^{i ξ log {α}_{k}}}{- 2 πξ} d ξ$$

corresponds to a Levy inversion formula. In the right side of formula (23),

$$( \frac{1}{Γ ( 1 + i ξ )} {\int }_{0}^{T} \{ y ( t ) - b \} {e}^{i ξ logt} dt )$$

is a characteristic function.

As shown in formula (23), in the present example embodiment, a formula derived from a Levy inversion formula, not a Fourier inversion formula, is used as a technique for reconstructing a discrete measure.

(Relationship Between Feature ν_{y-b}((αₖ₋₁, αₖ]) and b, ξₖ, and βₖ)

In formula (23) above, in a case where (αₖ−αₖ₋₁) is sufficiently less than αₖ₋₁ and the range c of integration is sufficiently wide, the value ν_{y-b}((αₖ₋₁, αₖ]) is an approximate value of

$${\sum }_{k : {α}_{k - 1} < k \leq {α}_{k}} {ξ}_{k} .$$

That is, the value ν_{y-b}((αₖ₋₁, αₖ]) is an approximate value of a sum of all ξₖ that satisfy αₖ₋₁<ξₖ≤αₖ.

In this case, assuming that positive numbers βₘᵢₙ and βₘₐₓ that satisfy βₘᵢₙ≤β₁< . . . <β_{K}≤βₘₐₓ are available, fixing divisions βₘᵢₙ=α₀<α₁< . . . <αₙ=βₘₐₓ with sufficiently small widths and calculating ν_{y-b}((αₖ₋₁, αₖ]) in accordance with formula (23) above for each k=1, . . . , n allows the following features of the signal y(t) to be extracted.

b,α₀,ν_{y-b}((α₀,α₁]), . . . ,αₙ₋₁,ν_{y-b}((αₙ₋₁,αₙ])

These features are equivalent to the features b, β, and ξₖ to be extracted as values characterizing the signal y(t). Because of the setting that the natural number K is unknown, n values αₖ, which are equivalent to βₖ, and n values ν_{y-b}((αₖ₋₁, αₖ]), which are equivalent to ξₖ, are determined, the number n being a sufficiently large set number. In other words, to compensate for the fact that K is unknown, n is set to a large number, so that there are a large number of features.

αₖ, which is artificially set regardless of the signal y(t), is not a feature per se, but characterizes the signal y(t) in the meaning that a coefficient corresponding to αₖ (equivalent to βₖ) is ν_{y-b}((αₖ₋₁, αₖ]) (equivalent to ξₖ). Thus, the signal y(t) can be represented with use of ξₖ and βₖ which are derived from ν_{y-b}((αₖ₋₁, αₖ]) calculated by the calculation section 11A and αₖ.

### (Effect of Feature Calculation Apparatus)

As described above, according to the present example embodiment, when reproducing an original discrete measure from a signal y(t) which is represented as a combination of a Laplace transform of the discrete measure and an offset b, use of a formula derived from a Levy inversion formula instead of a Fourier inversion formula makes it possible to reproduce the discrete measure.

Further, in the feature calculation apparatus 1A in accordance with the present example embodiment, a configuration is employed in which the offset b included in the signal y(t) is known and the calculation section 11A calculates the value ν_{y-b}((αₖ₋₁, αₖ]) with use of formula (23) above. As such, the feature calculation apparatus 1A in accordance with the present example embodiment provides not only the effects provided by the feature calculation apparatus 1 in accordance with the first example embodiment but also the effect of making it possible to reproduce a discrete measure having, in a Laplace transform, a signal y(t) in which the offset b is known.

Further, in the feature calculation apparatus 1A in accordance with the present example embodiment, a configuration is employed in which the output section 12A outputs, as a feature of the signal y(t), information indicative of the value ν_{y-b}((αₖ₋₁, αₖ]) and outputs, as additional information, information indicative of the interval (αₖ₋₁, αₖ]. As such, the feature calculation apparatus 1A in accordance with the present example embodiment provides not only the effects provided by the feature calculation apparatus 1 in accordance with the first example embodiment but also the effect of making it possible to both: reproduce a discrete measure having, in a Laplace transform, a signal y(t) in which the offset b is known; and output information indicative of the interval (αₖ₋₁, αₖ].

### Third Example Embodiment

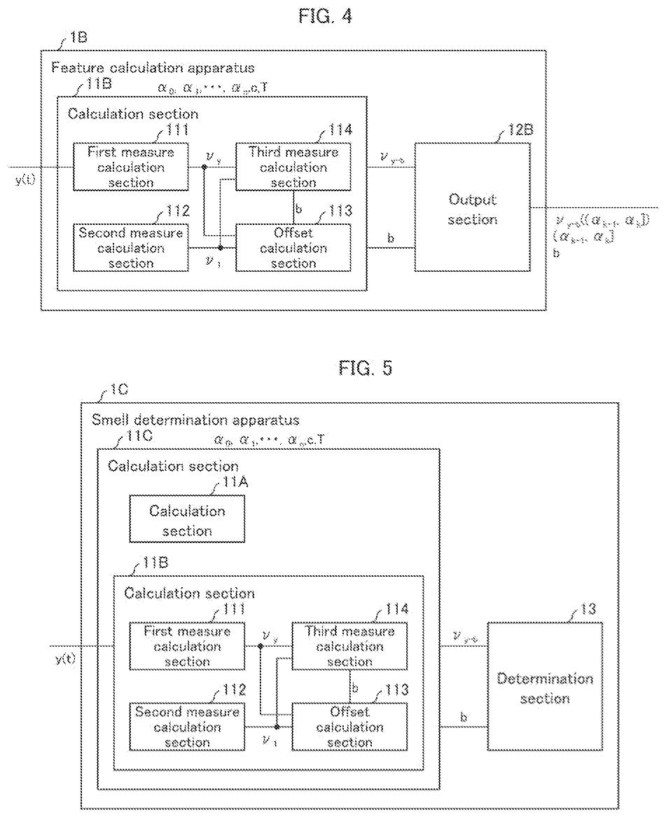

The following will discuss in detail a third example embodiment of the present invention, with reference to drawings. FIG. 4 is a block diagram illustrating a configuration of a feature calculation apparatus 1B in accordance with the present example embodiment. The feature calculation apparatus 1B includes a calculation section 11B and an output section 12B.

The calculation section 11B calculates, for each of n consecutive intervals (α₀, α₁], (α₁, α₂], . . . , (αₙ₋₁, αₙ], a value ν_{y-b}((αₖ₋₁, αₖ]) as a feature of a signal y(t) represented by formula (21) above. Note that an offset b included in formula (21) representing the signal y(t) is unknown.

The calculation section 11B includes a first measure calculation section 111, a second measure calculation section 112, an offset calculation section 113, and a third measure calculation section 114. The first measure calculation section 111 calculates a value ν_{y}((αₖ₋₁, αₖ]) with use of formula (24) below:

$${v}_{y} ( ( {α}_{k - 1} , {α}_{k} ] ) = 2 {α}_{k - 1} \times Im [ {\int }_{0}^{c} ( \frac{1}{Γ ( 1 + i ξ )} {\int }_{0}^{T} y ( t ) {e}^{i ξ logt} dt ) \frac{{e}^{i {ξ log α}_{k - 1}} - {e}^{i ξ log {α}_{k}}}{- 2 πξ} d ξ ] \qquad ( 24 )$$

where Γ(⋅) is a complex gamma function, Im(⋅) is an imaginary part of ⋅, log is a natural logarithmic function, π is a ratio of a circumference of a circle to a diameter thereof, i is an imaginary unit, c is a positive number, and T is a positive number representing measurement time at which the signal y(t) is measured.

The second measure calculation section 112 calculates a value ν₁((αₖ₋₁, αₖ]) with use of formula (25) below.

$${v}_{1} ( ( {α}_{k - 1} , {α}_{k} ] ) = 2 {α}_{k - 1} \times Im [ {\int }_{0}^{c} ( \frac{1}{Γ ( 1 + i ξ )} {\int }_{0}^{T} {e}^{i ξ log t} dt ) \frac{{e}^{i ξ log {α}_{k - 1}} - {e}^{i ξ log {α}_{k}}}{- 2 πξ} d ξ ] \qquad ( 25 )$$

The offset calculation section 113 calculates the offset b with use of formula (26) below.

$$b = - \frac{\begin{matrix}{\int }_{0}^{T} \{ Y ( t ) - {\sum }_{k = 1}^{n} {v}_{y} ( ( {α}_{k - 1} , {α}_{k} ] ) {e}^{{α}_{k} t} \} \\ \{ {\sum }_{k = 1}^{n} {v}_{1} ( ( {α}_{k - 1} , {α}_{k} ] ) {e}^{- {α}_{k} t} - 1 \} dt\end{matrix}}{{{\int }_{0}^{T} \{ {\sum }_{k = 1}^{n} ⅆ {v}_{1} ( ( {α}_{k - 1} , {α}_{k} ] ) {e}^{- {α}_{k} t} - 1 \}}^{2} dt} \qquad ( 26 )$$

With respect to the offset b calculated by the offset calculation section 113, the following features of the signal y(t) are extracted.

b,α₀,ν_{y}((α₀,α₁])−bν₁((α₀,α₁]), . . . ,

αₙ₋₁,ν_{y}((αₙ₋₁,αₙ])−bν₁((αₙ₋₁,αₙ])

Assuming here that f(t)=y(t)−b, the following equation:

ν_{y-b}((αₖ₋₁,αₖ])=ν_{y}((αₖ₋₁,αₖ])−bν₁((αₖ₋₁,αₖ]) (27)

is met due to linearity of the corresponding

f→ν_{f}((A,B])

. That is, the ultimate output is a value similar to that in a case in which the offset b is known. As such, the third measure calculation section 114 calculates the value ν_{y-b}((αₖ₋₁, αₖ]) with use of formula (27) above.

The information used in the calculation process carried out by the calculation section 11B can be, for example, (i) measurement time T, (ii) a range c of integration, (iii) a lower limit βₘᵢₙ of a coefficient of exponential decrease, (iv) an upper limit βₘₐₓ of the coefficient of exponential decrease, and (v) values α₀, α₁, . . . , αₙ, by which divided intervals of a range where the coefficient of exponential decrease exists are determined. These pieces of information can each be information (default value) preset in the feature calculation apparatus 1B. Alternatively, the pieces of information may be inputted by a user with use of an input apparatus and then used by the calculation section 11B. Alternatively, the feature calculation apparatus 1B may calculate at least part of the pieces of information.

The output section 12B outputs, as a feature of the signal y(t), information indicative of the value ν_{y-b}((αₖ₋₁, αₖ]), outputs, as additional information, information indicative of the interval (α₀, α₁], and outputs, as offset information, information indicative of the offset b.

As described above, according to the present example embodiment, when reproducing an original discrete measure from a signal y(t) which is represented as a combination of a Laplace transform of the discrete measure and an offset b, use of a formula derived from a Levy inversion formula instead of a Fourier inversion formula makes it possible to reproduce the discrete measure.

In the feature calculation apparatus 1B in accordance with the present example embodiment, a configuration is employed in which the offset b included in the signal y(t) is unknown and the calculation section 11B includes the first measure calculation section 111 that calculates the value ν_{y}((αₖ₋₁, αₖ]) with use of formula (24) above, the second measure calculation section 112 that calculates the value ν₁((αₖ₋₁, αₖ]) with use of formula (25) above, the offset calculation section 113 that calculates the offset b with use of formula (26) above, and the third measure calculation section 114 that calculates the value ν_{y-b}((αₖ₋₁, αₖ]) with use of formula (27) above. As such, the feature calculation apparatus 1B in accordance with the present example embodiment provides not only the effects provided by the feature calculation apparatus 1 in accordance with the first example embodiment but also the effect of making it possible to calculate a feature of a signal y(t) in which the offset b is unknown.

Further, in the feature calculation apparatus 1B in accordance with the present example embodiment, a configuration is employed in which the output section 12B is provided, the output section 12B outputting, as a feature of the signal y(t), information indicative of the value ν_{y-b}((αₖ₋₁, αₖ]), outputting, as additional information, information indicative of the interval (αₖ₋₁, αₖ], and outputting, as offset information, information indicative of the offset b. As such, the feature calculation apparatus 1B in accordance with the present example embodiment provides not only the effects provided by the feature calculation apparatus 1 in accordance with the first example embodiment but also the effect of making it possible to output information indicative of an interval (α₀, α₁] and information indicative of an offset b.

### Fourth Example Embodiment

The following will discuss in detail a fourth example embodiment of the present invention, with reference to drawings. Note that any constituent element that is identical in function to a constituent element described in any one(s) of the first to third example embodiments will be given the same reference numeral, and a description thereof will not be repeated.

### (Configuration of Smell Determination Apparatus)

FIG. 5 is a block diagram illustrating a configuration of a smell determination apparatus 1C in accordance with the present example embodiment. The smell determination apparatus 1C is an apparatus which carries out determination on a smell on the basis of a signal y(t) outputted from a smell sensor. The smell sensor is, for example, a membrane-type surface stress (MSS) sensor. With use of the MSS sensor, an electric signal value at the time when molecules of a smell gas are adsorbed on or desorbed from a membrane is measured. Note that the smell sensor is not limited to an MSS sensor but can be other sensors.

The smell determination apparatus 1C includes a calculation section 11C and a determination section 13. The determination section 13 is an example of a determination apparatus in this specification. The calculation section 11C includes a calculation section 11A and a calculation section 11B. The calculation section 11C calculates, for each of n consecutive intervals (α₀, α₁], (α₁, α₂], . . . , (αₙ₋₁, αₙ], a value ν_{y-b}((αₖ₋₁, αₖ]) as a feature of a signal y(t) represented by formula (21) above. At this time, the calculation section 11C determines whether the offset b is unknown or known, and in a case where the offset b is unknown, the calculation section 11C calculates ν_{y-b}((αₖ₋₁, αₖ]) by carrying out the above-described calculation process carried out by the calculation section 11A in accordance with the second example embodiment. In a case where the offset b is known, the calculation section 11C calculates ν_{y-b}((αₖ₋₁, αₖ]) by carrying out the above-described calculation process carried out by the calculation section 11B in accordance with the third example embodiment.

In the present example embodiment, the calculation section 11C determines that the offset b is known in a case where a differential of the signal y(t) is not more than a predetermined threshold, and determines that the offset b is unknown in a case where the differential of the signal y(t) is more than predetermined threshold. In other words, the calculation section 11C calculates the value ν_{y-b}((αₖ₋₁, αₖ]) with use of formula (23) above in a case where the differential of the signal y(t) is not more than the predetermined threshold. In a case where the differential is more than the threshold, the calculation section 11C calculates the value ν_{y-b}((αₖ₋₁, αₖ]) by the first measure calculation section 111, the second measure calculation section 112, the offset calculation section 113, and the third measure calculation section 114. The method of setting the information (measurement time T, a range c of integration, a lower limit βₘᵢₙ of a coefficient of exponential decrease, an upper limit βₘₐₓ of the coefficient of exponential decrease, the values α₀, α₁, . . . , αₙ by which divided intervals of a range where the coefficient of exponential decrease exists are determined, etc.) used in the calculation process carried out by the calculation section 11C is similar to the method described in the second example embodiment above.

The determination section 13 determines a type of the smell or a type of a source of the smell on the basis of the feature calculated by the calculation section 11C. For example, the determination section 13 may carry out the determination of a type by comparing the feature calculated by the calculation section 11C with a plurality of features registered in a predetermined database. Alternatively, for example, the determination section 13 may determine the type of the smell by inputting a feature calculated by the calculation section 11B to a trained model that receives input of a feature of the signal y(t) and outputs a type of a smell. In this case, the trained model is a trained model that has been constructed by machine learning with use of training data including a feature of a signal y(t) and a label indicative of a type of a smell. The technique of machine learning of the trained model is not limited. For example, a decision tree-based technique, a technique of linear regression, or a technique of neural network can be used, and two or more of these techniques can be used. Note that the technique by which the determination section 13 carried out the determination process is not limited to the above-described examples, and the determination section 13 can carry out the determination process by other techniques.

### (Flow of Smell Determination Method)

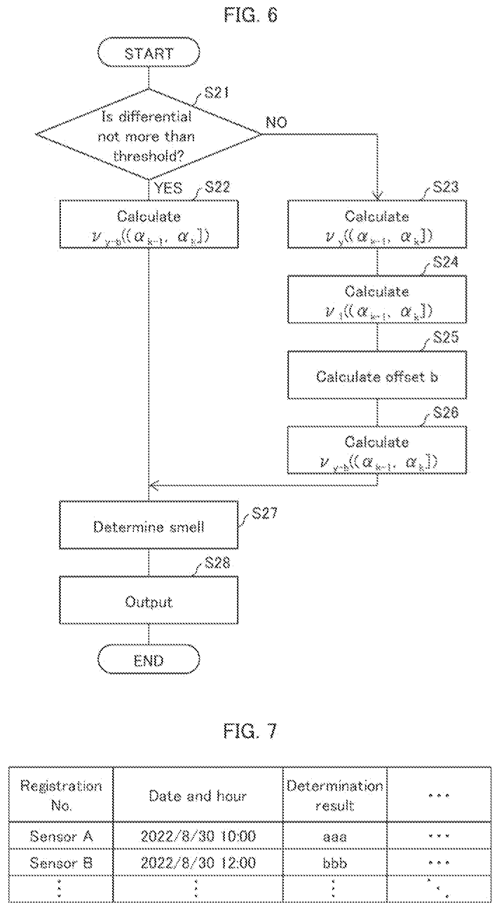

FIG. 6 is a flowchart illustrating a flow of a smell determination method carried out by the smell determination apparatus 1C. At S21, the calculation section 11C determines whether or not a differential of the signal y(t) is not more than a predetermined threshold. In a case where the differential of the signal y(t) is not more than the predetermined threshold (YES at step S21), the calculation section 11C proceeds to the process of step S22. In a case where the differential of the signal y(t) is more than the predetermined threshold (NO at step S21), the calculation section 11C proceeds to the process of step S23.

At S22, the calculation section 11C calculates, for each of n consecutive intervals (α₀, α₁], (α₁, α₂], . . . , (αₙ₋₁, αₙ], a value ν_{y-b}((αₖ₋₁, αₖ]) with use of formula (23) above. The calculation process carried out by the calculation section 11C at step S22 is similar to the calculation process carried out by the calculation section 11A in accordance with the second example embodiment described above. At this time, the calculation section 11C employs a value of y(t) at final time as an approximate value of the offset b and calculates the value ν_{y-b}((αₖ₋₁, αₖ]) with use of formula (23) above.

At S23, the calculation section 11C calculates the value ν_{y}((αₖ₋₁, αₖ]) with use of formula (24) above. The calculation process carried out by the calculation section 11C at step S23 is similar to the calculation process carried out by the first measure calculation section 111 in accordance with the third example embodiment described above.

At S24, the calculation section 11C calculates the value ν₁((αₖ₋₁, αₖ]) with use of formula (25) above. The calculation process carried out by the calculation section 11C at step S24 is similar to the calculation process carried out by the second measure calculation section 112 in accordance with the third example embodiment described above.

At S25, the calculation section 11C calculates the offset b with use of formula (26) above. The calculation process carried out by the calculation section 11C at step S25 is similar to the calculation process carried out by the offset calculation section 113 in accordance with the third example embodiment described above.

At S26, the calculation section 11C calculates the value ν_{y-b}((αₖ₋₁, αₖ]) with use of formula (27) above. The calculation process carried out by the calculation section 11C at step S26 is similar to the calculation process carried out by the third measure calculation section 114 in accordance with the third example embodiment described above.

At S27, the determination section 13 determines, on the basis of the feature calculated by the calculation section 11C, a type of smell represented by the signal y(t). At S28, the determination section 13 outputs information indicative of at least one of the feature calculated by the calculation section 11C and a result of determination by the determination section 13. For example, the determination section 13 can output a type of a smell, which is a determination result. For example, the determination section 13 can output parameters b, ξₖ, and βₖ of the signal y(t) determined on the basis of the feature calculated by the calculation section 11C. Further, the determination section 13 can output and display an output value actually outputted from a sensor and inputted to the smell determination apparatus 1C and the signal y(t) calculated on the basis of the feature calculated by the calculation section 11C.

FIG. 7 is a diagram illustrating an example of display of a result of determination outputted at step S28. In the example illustrated in FIG. 7, identification information for identifying a sensor, the date and hour when the determination section 13 carried out a determination process, a result of determination by the determination section 13, and the like are displayed so as to be associated with one another. Note that the information indicative of the determination result outputted by the determination section 13 is not limited to the example illustrated in FIG. 7. For example, the determination section 13 can output and display parameters b, ξₖ, and βₖ of the signal y(t).

### (Specific Example of Actual Measured Value and Calculation Result)

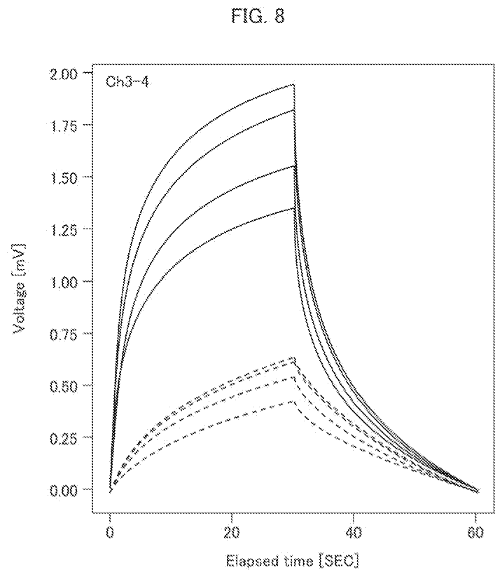

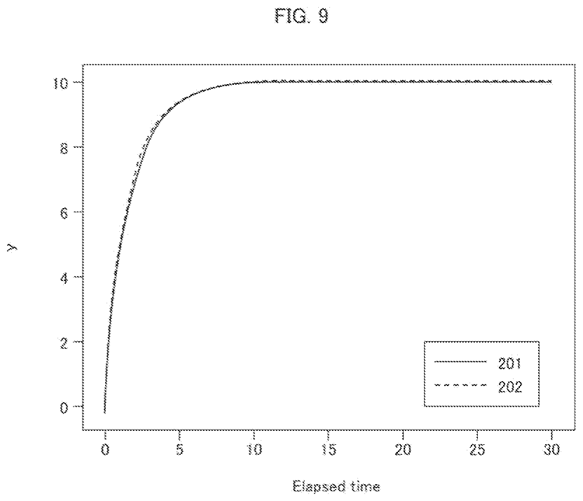

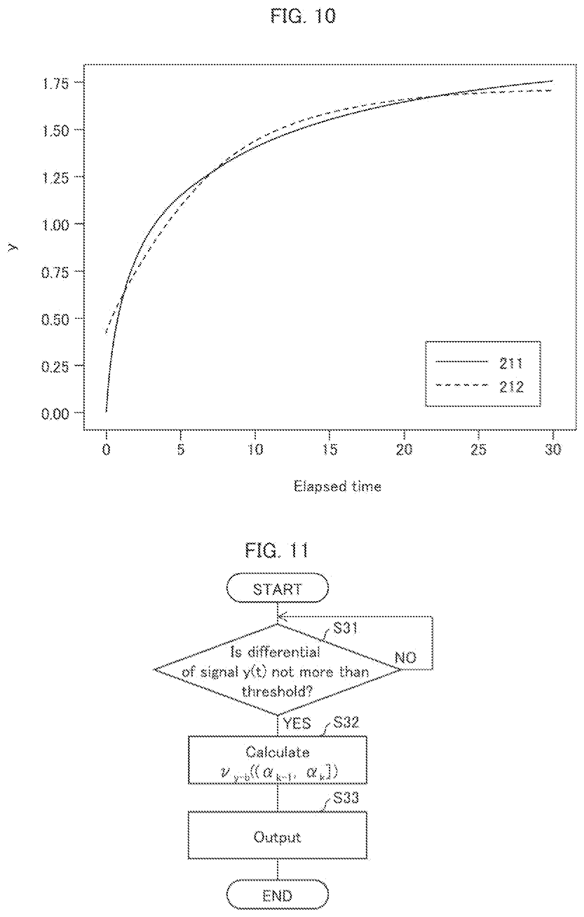

FIG. 8 is a diagram illustrating an example of a signal y(t) measured by an MSS sensor. In FIG. 8, the horizontal axis represents elapsed time, and the vertical axis represents a signal value. FIGS. 9 and 10 are diagrams each illustrating an example of a signal y(t) represented with use of a feature calculated by the calculation section 11C. FIG. 9 shows original data 201 artificially generated and a signal y(t) 202 represented with use of a feature calculated by the calculation section 11C in accordance with the present example embodiment with respect to the original data 201. The original data 201 is data represented as y(t)=10−7e^{-0.5t}−3e^{-0.75t}.

FIG. 10 shows actual measurement data 211 measured by an MSS sensor and a signal y(t) 212 represented with use of a feature calculated by the calculation section 11C. As shown in FIGS. 9 and 10, it is possible to represent a signal y(t) with use of a feature calculated by the calculation section 11C.

### (Effect of Smell Determination Apparatus)

As described above, the smell determination apparatus 1C in accordance with the present example embodiment calculates a feature of a signal y(t) outputted from a smell sensor. As such, according to the present example embodiment, it is possible to calculate a feature of a signal y(t) representing a smell and measured by the smell sensor.

Further, the smell determination apparatus 1C in accordance with the present example embodiment includes the determination section 13 which determines a type of a smell or a type of a source of the smell on the basis of the feature calculated by the calculation section 11C. As such, the smell determination apparatus 1C in accordance with the present example embodiment makes it possible to determine a type of a smell or a type a source of the smell, by reproducing an original discrete measure from a Laplace transform of the discrete measure.

Further, the calculation section 11C in accordance with the present example embodiment calculates the value ν_{y-b}((αₖ₋₁, αₖ]) with use of formula (23) above in a case where the differential of the signal y(t) is not more than a predetermined threshold. In a case where the differential is more than the threshold, the calculation section 11C calculates the value ν_{y-b}((αₖ₋₁, αₖ]) by the first measure calculation section 111, the second measure calculation section 112, the offset calculation section 113, and the third measure calculation section 114. By calculating v on the assumption that the offset b is known in a case where the differential is not more than the threshold, it is possible to increase the accuracy of calculation of the feature.

### Variation

In the above-described second example embodiment, the calculation section 11A may start the process of calculating a feature until observation of the signal y(t) finds that the signal y(t) has come into a steady state (flat state). In other words, the calculation section 11A may start the process of calculating a feature in a case where a differential of the signal y(t) is not more than a predetermined threshold.

FIG. 11 is a flowchart illustrating a flow of a feature calculation method in accordance with the present variation. At S31, the calculation section 11A determines whether or not a differential of the signal y(t), which is to be analyzed, is not more than a predetermined threshold. In a case where the differential is not more than the threshold (YES at step S31), the calculation section 11A proceeds to the process of step S32. In a case where the differential is more than the threshold (NO at step S31), the calculation section 11A waits until the differential is not more than the threshold.

At step S32, the calculation section 11A calculates a value ν_{y-b}((αₖ₋₁, αₖ]). At this time, the calculation section 11A regards a limit value of the signal y(t), which limit value is estimated from the signal y(t), to be an offset b, regards the offset b to be known, and calculates the value ν_{y-b}((αₖ₋₁, αₖ]) with use of formula (23) above. At step S33, the output section 12A outputs the feature calculated by the calculation section 11A. According to the present example aspect, the use of the signal y(t) observed until the signal y(t) comes into a steady state (flat state) makes it possible to increase the accuracy of inverse transform.

### Software Implementation Example

Some or all of the functions of each of the feature calculation apparatuses 1, 1A, and 1B and the smell determination apparatus 1C (hereinafter, referred to as “feature calculation apparatus 1 etc.”) may be realized by hardware such as an integrated circuit (IC chip) or may be alternatively realized by software.

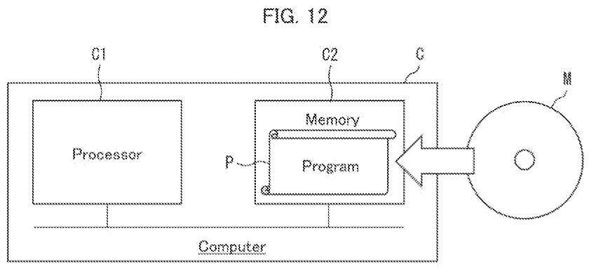

In the latter case, each of the feature calculation apparatus 1 etc. is realized by, for example, a computer that executes instructions of a program that is software realizing the foregoing functions. FIG. 12 illustrates an example of such a computer (hereinafter, referred to as “computer C”). The computer C includes at least one processor C1 and at least one memory C2. The memory C2 stores a program P for causing the computer C to function as the feature calculation apparatus 1 etc. In the computer C, the processor C1 reads the program P from the memory C2 and executes the program P, so that the functions of the feature calculation apparatus 1 etc. are realized.

As the processor C1, for example, it is possible to use a central processing unit (CPU), a graphic processing unit (GPU), a digital signal processor (DSP), a micro processing unit (MPU), a floating point number processing unit (FPU), a physics processing unit (PPU), a tensor processing unit (TPU), a quantum processor, a microcontroller, or a combination of these. The memory C2 can be, for example, a flash memory, a hard disk drive (HDD), a solid state drive (SSD), or a combination of these.

Note that the computer C can further include a random access memory (RAM) in which the program P is loaded when the program P is executed and in which various kinds of data are temporarily stored. The computer C can further include a communication interface for carrying out transmission and reception of data with other apparatuses. The computer C can further include an input-output interface for connecting input-output apparatuses such as a keyboard, a mouse, a display, and a printer.

The program P can be stored in a non-transitory tangible storage medium M which is readable by the computer C. The storage medium M can be, for example, a tape, a disk, a card, a semiconductor memory, a programmable logic circuit, or the like. The computer C can obtain the program P via the storage medium M. The program P can be transmitted via a transmission medium. The transmission medium can be, for example, a communications network, a broadcast wave, or the like. The computer C can obtain the program P also via such a transmission medium.

### Additional Remark 1

The present invention is not limited to the foregoing example embodiments, but may be altered in various ways by a skilled person within the scope of the claims. For example, the present invention also encompasses, in its technical scope, any example embodiment derived by appropriately combining technical means disclosed in the foregoing example embodiments.

### Additional Remark 2

The whole or part of the example embodiments disclosed above can be described as, but not limited to, the following supplementary notes.

### Supplementary Note 1

A feature calculation apparatus, including a calculation means that calculates, for each of n consecutive intervals (α₀, α₁], (α₁, α₂], . . . , (αₙ₋₁, αₙ], a value ν_{y-b}((αₖ₋₁, αₖ]) as a feature of a signal y(t) approximated by formula (31) below and including an offset b, the value ν_{y-b}((αₖ₋₁, αₖ]) being approximated by formula (32) below:

$$\begin{matrix}y ( t ) = b + {\sum }_{k = 1}^{K} {ξ}_{k} {e}^{- {β}_{k} t} & ( 31 ) \\ {v}_{y - b} ( ( {α}_{k - 1} , {α}_{k} ] ) = {\sum }_{k : {α}_{k - 1} < k \leq {α}_{k}} {ξ}_{k} & ( 32 )\end{matrix}$$

where K and n are each a natural number, b and ξ₁, ξ₂, . . . , are each a real number, β₁, β₂, . . . , β_{K} are each a positive number, and α₀, α₁, . . . , αₙ are each a positive number that satisfies βₘᵢₙ=α₀<α₁< . . . <αₙ=βₘₐₓ where βₘᵢₙ≤min{β₁, β₂, . . . , β_{K}} and βₘₐₓ≥max{β₁, β₂, . . . , β_{K}}.

### Supplementary Note 2

The feature calculation apparatus according to supplementary note 1, wherein:
the offset b included in the signal y(t) is known; and
the calculation means calculates the value ν_{y-b}((αₖ₋₁, αₖ]) with use of formula (33) below:

$${v}_{y - b} ( ( {α}_{k - 1} , {α}_{k} ] ) = 2 {α}_{k - 1} \times Im [ {\int }_{0}^{c} ( \frac{1}{Γ ( 1 + i ξ )} {\int }_{0}^{T} \{ Y ( t ) - b \} {e}^{- i ξ log t} dt ) \frac{{e}^{i ξ log {α}_{k - 1}} - {e}^{i ξ log {α}_{k}}}{- 2 πξ} d ξ ] \qquad ( 33 )$$

where Γ(⋅) is a complex gamma function, Im(⋅) is an imaginary part of ⋅, log is a natural logarithmic function, π is a ratio of a circumference of a circle to a diameter thereof, i is an imaginary unit, c is a positive number, and T is a positive number representing measurement time at which the signal y(t) is measured.

### Supplementary Note 3

The feature calculation apparatus according to supplementary note 2, further including an output means that outputs, as the feature of the signal y(t), information indicative of the value ν_{y-b}((αₖ₋₁, αₖ]) and outputs, as additional information, information indicative of an interval (αₖ₋₁, αₖ].

### Supplementary Note 4

The feature calculation apparatus according to supplementary note 1, wherein:
the offset b included in the signal y(t) is unknown; and
the calculation means includes a first measure calculation means that calculates a value ν_{y}((αₖ₋₁, αₖ]) with use of formula (34) below, a second measure calculation means that calculates a value ν₁((αₖ₋₁, αₖ]) with use of formula (35) below, an offset calculation means that calculates the offset b with use of formula (36) below, and a third measure calculation means that calculates the value ν_{y-b}((αₖ₋₁, αₖ]) with use of formula (37) below:

$$\begin{matrix}{v}_{y} ( ( {α}_{k - 1} , {α}_{k} ] ) = 2 {α}_{k - 1} \times Im [ {\int }_{0}^{c} ( \frac{1}{Γ ( 1 + i ξ )} {\int }_{0}^{T} {e}^{i ξ log t} dt ) \frac{{e}^{i ξ log {α}_{k - 1}} - {e}^{i ξ log {α}_{k}}}{- 2 πξ} d ξ ] & ( 34 ) \\ {v}_{1} ( ( {α}_{k - 1} , {α}_{k} ] ) = 2 {α}_{k - 1} \times Im [ {\int }_{0}^{c} ( \frac{1}{Γ ( 1 + i ξ )} {\int }_{0}^{T} {e}^{i ξ log t} dt ) \frac{{e}^{i ξ log {α}_{k - 1}} - {e}^{i ξ log {α}_{k}}}{- 2 πξ} d ξ ] & ( 35 )\end{matrix}$$

$$\begin{matrix}b = - \frac{\begin{matrix}{\int }_{0}^{T} \{ Y ( t ) - {\sum }_{k = 1}^{n} {v}_{y} ( ( {α}_{k - 1} , {α}_{k} ] ) {e}^{{α}_{k} t} \} \\ \{ {\sum }_{k = 1}^{n} {v}_{1} ( ( {α}_{k - 1} , {α}_{k} ] ) {e}^{- {α}_{k} t} - 1 \} dt\end{matrix}}{{{\int }_{0}^{T} \{ {\sum }_{k = 1}^{n} ⅆ {v}_{1} ( ( {α}_{k - 1} , {α}_{k} ] ) {e}^{- {α}_{k} t} - 1 \}}^{2} dt} & ( 36 ) \\ {v}_{y - b} ( ( {α}_{k - 1} , {α}_{k} ] ) = {v}_{y} ( ( {α}_{k - 1} , {α}_{k} ] ) - {bv}_{1} ( ( {α}_{k - 1} , {α}_{k} ] ) & ( 37 )\end{matrix}$$

where Γ(⋅) is a complex gamma function, Im(⋅) is an imaginary part of ⋅, log is a natural logarithmic function, π is a ratio of a circumference of a circle to a diameter thereof, i is an imaginary unit, c is a positive number, and T is a positive number representing measurement time at which the signal y(t) is measured.

### Supplementary Note 5

The feature calculation apparatus according to supplementary note 4, further including an output means that outputs, as a feature of the signal y(t), information indicative of the value ν_{y-b}((αₖ₋₁, αₖ]), outputs, as additional information, information indicative of the interval (α₀, α₁], and outputs, as offset information, information indicative of the offset b.

### Supplementary Note 6

The feature calculation apparatus according to any one of supplementary notes 1 to 5, wherein the signal y(t) is a signal outputted from a smell sensor.

### Supplementary Note 7

A smell determination apparatus, including the feature calculation apparatus according to supplementary note 6 and a determination apparatus that determines, on the basis of the feature calculated by the feature calculation apparatus, a type of a smell or a type of a source of a smell.

### Supplementary Note 8

The feature calculation apparatus according to supplementary note 4, wherein the calculation means
calculates the value ν_{y-b}((αₖ₋₁, αₖ]) with use of formula (38) below in a case where a differential of the signal y(t) is not more than a predetermined threshold, and
calculates the value ν_{y-b}((αₖ₋₁, αₖ]) by the first measure calculation means, the second measure calculation means, the offset calculation means, and the third measure calculation means in a case where the differential is more than the predetermined threshold:

$${v}_{y - b} ( ( {α}_{k - 1} , {α}_{k} ] ) = 2 {α}_{k - 1} \times Im [ {\int }_{0}^{c} ( \frac{1}{Γ ( 1 + i ξ )} {\int }_{0}^{T} \{ Y ( t ) - b \} {e}^{- i ξ log t} dt ) \frac{{e}^{i ξ log {α}_{k - 1}} - {e}^{i ξ log {α}_{k}}}{- 2 πξ} d ξ ] \qquad ( 38 )$$

where Γ(⋅) is a complex gamma function, Im(⋅) is an imaginary part of ⋅, log is a natural logarithmic function, π is a ratio of a circumference of a circle to a diameter thereof, i is an imaginary unit, c is a positive number, and T is a positive number representing measurement time at which the signal y(t) is measured.

### Supplementary Note 9

A feature calculation method, including calculating, by at least one processor and for each of n consecutive intervals (α₀, α₁], (α₁, α₂], . . . , (αₙ₋₁, αₙ], a value ν_{y-b}((αₖ₋₁, αₖ]) as a feature of a signal y(t) approximated by formula (39) below and including an offset b, the value ν_{y-b}((αₖ₋₁, αₖ]) being approximated by formula (40) below:

$$\begin{matrix}y ( t ) = b + {\sum }_{k = 1}^{K} {ξ}_{k} {e}^{- {β}_{k} t} & ( 39 ) \\ {v}_{y - b} ( ( {α}_{k - 1} , {α}_{k} ] ) = {\sum }_{k : {α}_{k - 1} < k \leq {α}_{k}} {ξ}_{k} & ( 40 )\end{matrix}$$

where K and n are each a natural number, b and ξ₁, ξ₂, . . . , ξ_{K} are each a real number, β₁, β₂, . . . , β_{K} are each a positive number, and α₀, α₁, . . . , αₙ are each a positive number that satisfies βₘᵢₙ=α₀<α₁< . . . <αₙ=βₘₐₓ where βₘᵢₙ≤min{β₁, β₂, . . . , β_{K}} and βₘₐₓ≥max{β₁, β₂, . . . , β_{K}}.

### Supplementary Note 10

A program for causing a computer to carry out a process of calculating, for each of n consecutive intervals α₀, (α₁], (α₁, α₂], . . . , (αₙ₋₁, αₙ], a value ν_{y-b}((αₖ₋₁, αₖ]) as a feature of a signal y(t) approximated by formula (41) below and including an offset b, the value ν_{y-b}((αₖ₋₁, αₖ]) being approximated by formula (42) below:

$$\begin{matrix}y ( t ) = b + {\sum }_{k = 1}^{K} {ξ}_{k} {e}^{- {β}_{k} t} & ( 41 ) \\ {v}_{y - b} ( ( {α}_{k - 1} , {α}_{k} ] ) = {\sum }_{k : {α}_{k - 1} < k \leq {α}_{k}} {ξ}_{k} & ( 42 )\end{matrix}$$

where K and n are each a natural number, b and ξ₁, ξ₂, . . . , ξ_{K} are each a real number, β₁, β₂, . . . , β_{K} are each a positive number, and α₀, α₁, . . . , αₙ are each a positive number that satisfies βₘᵢₙ=α₀<α₁< . . . <αₙ=βₘₐₓ where βₘᵢₙ≤min{β₁, β₂, . . . , β_{K}} and βₘₐₓ≥max{β₁, β₂, . . . , β_{K}}.

### Supplementary Note 11

A feature calculation apparatus, including at least one processor, the at least one processor carrying out a calculation process of calculating, for each of n consecutive intervals (α₀, α₁], (α₁, α₂], . . . , (αₙ₋₁, αₙ], a value ν_{y-b}((αₖ₋₁, αₖ]) as a feature of a signal y(t) approximated by formula (43) below and including an offset b, the value ν_{y-b}((αₖ₋₁, αₖ]) being approximated by formula (44) below:

$$\begin{matrix}y ( t ) = b + {\sum }_{k = 1}^{K} {ξ}_{k} {e}^{- {β}_{k} t} & ( 43 ) \\ {v}_{y - b} ( ( {α}_{k - 1} , {α}_{k} ] ) = {\sum }_{k : {α}_{k - 1} < k \leq {α}_{k}} {ξ}_{k} & ( 44 )\end{matrix}$$

where K and n are each a natural number, b and ξ₁, ξ₂, . . . , ξ_{K} are each a real number, β₁, β₂, . . . , β_{K} are each a positive number, and α₀, α₁, . . . , αₙ are each a positive number that satisfies βₘᵢₙ=α₀<α₁< . . . <αₙ=βₘₐₓ where βₘᵢₙ≤min{β₁, β₂, . . . , β_{K}} and βₘₐₓ≥max{β₁, β₂, . . . , β_{K}}.

Note that the feature calculation apparatus may further include a memory, which may store a program for causing the at least one processor to carry out the calculation process. The program can be stored in a computer-readable non-transitory tangible storage medium.

### REFERENCE SIGNS LIST

1, 1A, 1B: Feature calculate apparatus
1C: Smell determination apparatus
11, 11A, 11B, 11C: Calculation section
12A, 12B: Output section
13: Determination section
111: First measure calculation section
112: Second measure calculation section
113: Offset calculation section
114: Third measure calculation section
S1: Feature calculation method

1. A feature calculation apparatus, comprising at least one processor, the at least one processor carrying out
a calculation process of calculating, for each of n consecutive intervals (α₀, α₁], (α₁, α₂], . . . , (αₙ₋₁, αₙ], a value ν_{y-b}((αₖ₋₁, αₖ]) as a feature of a signal y(t) approximated by formula (1) below and including an offset b, the value ν_{y-b}((αₖ₋₁, αₖ]) being approximated by formula (2) below:
$$\begin{matrix}y ( t ) = b + {\sum }_{k = 1}^{K} {ξ}_{k} {e}^{- {β}_{k} t} & ( 1 ) \\ {v}_{y - b} ( ( {α}_{k - 1} , {α}_{k} ] ) = {\sum }_{k : {α}_{k - 1} < k \leq {α}_{k}} {ξ}_{k} & ( 2 )\end{matrix}$$
where K and n are each a natural number, b and ξ₁, ξ₂, . . . , ξ_{K} are each a real number, β₁, β₂, . . . , β_{K} are each a positive number, and α₀, α₁, . . . , αₙ are each a positive number that satisfies βₘᵢₙ=α₀<α₁< . . . <αₙ=βₘₐₓ where βₘᵢₙ≤min{β₁, β₂, . . . , β_{K}} and βₘₐₓ≥max{β₁, β₂, . . . , β_{K}}.

2. The feature calculation apparatus according to claim 1, wherein:
the offset b included in the signal y(t) is known; and
in the calculation process, the at least one processor calculates the value ν_{y-b}((αₖ₋₁, αₖ]) with use of formula (3) below:
$${v}_{y - b} ( ( {α}_{k - 1} , {α}_{k} ] ) = 2 {α}_{k - 1} \times Im [ {\int }_{0}^{c} ( \frac{1}{Γ ( 1 + i ξ )} {\int }_{0}^{T} \{ Y ( t ) - b \} {e}^{- i ξ log t} dt ) \frac{{e}^{i ξ log {α}_{k - 1}} - {e}^{i ξ log {α}_{k}}}{- 2 πξ} d ξ ] \qquad ( 3 )$$
where Γ(⋅) is a complex gamma function, Im(⋅) is an imaginary part of ⋅, log is a natural logarithmic function, π is a ratio of a circumference of a circle to a diameter thereof, i is an imaginary unit, c is a positive number, and T is a positive number representing measurement time at which the signal y(t) is measured.

3. The feature calculation apparatus according to claim 2, wherein the at least one processor further carries out an output process of outputting, as the feature of the signal y(t), information indicative of the value ν_{y-b}((αₖ₋₁, αₖ]) and outputting, as additional information, information indicative of an interval (αₖ₋₁, αₖ].

4. The feature calculation apparatus according to claim 1, wherein:
the offset b included in the signal y(t) is unknown; and
the calculation process includes a first measure calculation process of calculating a value ν_{y}((αₖ₋₁, αₖ]) with use of formula (4) below, a second measure calculation process of calculating a value ν₁((αₖ₋₁, αₖ]) with use of formula (5) below, an offset calculation process of calculating the offset b with use of formula (6) below, and a third measure calculation process of calculating the value ν_{y-b}((αₖ₋₁, αₖ]) with use of formula (7) below:
$$\begin{matrix}{v}_{y} ( ( {α}_{k - 1} , {α}_{k} ] ) = 2 {α}_{k - 1} \times Im [ {\int }_{0}^{c} ( \frac{1}{Γ ( 1 + i ξ )} {\int }_{0}^{T} {e}^{i ξ log t} dt ) \frac{{e}^{i ξ log {α}_{k - 1}} - {e}^{i ξ log {α}_{k}}}{- 2 πξ} d ξ ] & ( 4 ) \\ {v}_{1} ( ( {α}_{k - 1} , {α}_{k} ] ) = 2 {α}_{k - 1} \times Im [ {\int }_{0}^{c} ( \frac{1}{Γ ( 1 + i ξ )} {\int }_{0}^{T} {e}^{i ξ log t} dt ) \frac{{e}^{i ξ log {α}_{k - 1}} - {e}^{i ξ log {α}_{k}}}{- 2 πξ} d ξ ] & ( 5 )\end{matrix}$$
$$\begin{matrix}b = - \frac{\begin{matrix}{\int }_{0}^{T} \{ Y ( t ) - {\sum }_{k = 1}^{n} {v}_{y} ( ( {α}_{k - 1} , {α}_{k} ] ) {e}^{{α}_{k} t} \} \\ \{ {\sum }_{k = 1}^{n} {v}_{1} ( ( {α}_{k - 1} , {α}_{k} ] ) {e}^{- {α}_{k} t} - 1 \} dt\end{matrix}}{{{\int }_{0}^{T} \{ {\sum }_{k = 1}^{n} ⅆ {v}_{1} ( ( {α}_{k - 1} , {α}_{k} ] ) {e}^{- {α}_{k} t} - 1 \}}^{2} dt} & ( 6 ) \\ {v}_{y - b} ( ( {α}_{k - 1} , {α}_{k} ] ) = {v}_{y} ( ( {α}_{k - 1} , {α}_{k} ] ) - {bv}_{1} ( ( {α}_{k - 1} , {α}_{k} ] ) & ( 7 )\end{matrix}$$
where Γ(⋅) is a complex gamma function, Im(⋅) is an imaginary part of ⋅, log is a natural logarithmic function, π is a ratio of a circumference of a circle to a diameter thereof, i is an imaginary unit, c is a positive number, and T is a positive number representing measurement time at which the signal y(t) is measured.

5. The feature calculation apparatus according to claim 4, wherein the at least one processor further carries out an output process of outputting, as the feature of the signal y(t), information indicative of the value ν_{y-b}((αₖ₋₁, αₖ]), outputting, as additional information, information indicative of the interval (α₀, α₁], and outputting, as offset information, information indicative of the offset b.

6. The feature calculation apparatus according to claim 1, wherein the signal y(t) is a signal outputted from a smell sensor.

7. The feature calculation apparatus according to claim 6, wherein the at least one processor further carries out a determination process of determining, on the basis of the feature calculated in the calculation process, a type of a smell or a type of a source of a smell.

8. The feature calculation apparatus according to claim 4, wherein, in the calculation process, the at least one processor
calculates the value ν_{y-b}((αₖ₋₁, αₖ]) with use of formula (8) below in a case where a differential of the signal y(t) is not more than a predetermined threshold, and
calculates the value ν_{y-b}((αₖ₋₁, αₖ]) by the first measure calculation process, the second measure calculation process, the offset calculation process, and the third measure calculation process in a case where the differential is more than the predetermined threshold:
$${v}_{y - b} ( ( {α}_{k - 1} , {α}_{k} ] ) = 2 {α}_{k - 1} \times Im [ {\int }_{0}^{c} ( \frac{1}{Γ ( 1 + i ξ )} {\int }_{0}^{T} \{ Y ( t ) - b \} {e}^{- i ξ log t} dt ) \frac{{e}^{i ξ log {α}_{k - 1}} - {e}^{i ξ log {α}_{k}}}{- 2 πξ} d ξ ] \qquad ( 8 )$$
where Γ(⋅) is a complex gamma function, Im(⋅) is an imaginary part of ⋅, log is a natural logarithmic function, π is a ratio of a circumference of a circle to a diameter thereof, i is an imaginary unit, c is a positive number, and T is a positive number representing measurement time at which the signal y(t) is measured.

9. A feature calculation method, comprising calculating, by at least one processor and for each of n consecutive intervals (α₀, α₁], (α₁, α₂], . . . , (αₙ₋₁, αₙ], a value ν_{y-b}((αₖ₋₁, αₖ]) as a feature of a signal y(t) approximated by formula (9) below and including an offset b, the value ν_{y-b}((αₖ₋₁, αₖ]) being approximated by formula (10) below:
$$\begin{matrix}y ( t ) = b + {\sum }_{k = 1}^{K} {ξ}_{k} {e}^{- {β}_{k} t} & ( 9 ) \\ {v}_{y - b} ( ( {α}_{k - 1} , {α}_{k} ] ) = {\sum }_{k : {α}_{k - 1} < k \leq {α}_{k}} {ξ}_{k} & ( 10 )\end{matrix}$$
where K and n are each a natural number, b and ξ₁, ξ₂, . . . , ξ_{K} are each a real number, β₁, β₂, . . . , β_{K} are each a positive number, and α₀, α₁, . . . , αₙ are each a positive number that satisfies βₘᵢₙ=α₀<α₁< . . . <αₙ=βₘₐₓ where βₘᵢₙ≤min{β₁, β₂, . . . , β_{K}} and βₘₐₓ≥βmax{β₁, β₂, . . . , β_{K}}.

10. A computer-readable non-transitory storage medium storing therein a program for causing a computer to carry out
a process of calculating, for each of n consecutive intervals (α₀, α₁], (α₁, α₂], . . . , (αₙ₋₁, αₙ], a value ν_{y-b}((αₖ₋₁, αₖ]) as a feature of a signal y(t) approximated by formula (11) below and including an offset b, the value ν_{y-b}((αₖ₋₁, αₖ]) being approximated by formula (12) below:
$$\begin{matrix}y ( t ) = b + {\sum }_{k = 1}^{K} {ξ}_{k} {e}^{- {β}_{k} t} & ( 11 ) \\ {v}_{y - b} ( ( {α}_{k - 1} , {α}_{k} ] ) = {\sum }_{k : {α}_{k - 1} < k \leq {α}_{k}} {ξ}_{k} & ( 12 )\end{matrix}$$
where K and n are each a natural number, b and ξ₁, ξ₂, . . . , ξ_{K} are each a real number, β₁, β₂, . . . , β_{K} are each a positive number, and α₀, α₁, . . . , αₙ are each a positive number that satisfies βₘᵢₙ=α₀<α₁< . . . <αₙ=βₘₐₓ where βₘᵢₙ≤min{β₁, β₂, . . . , β_{K}} and βₘₐₓ≥max{β₁, β₂, . . . , β_{K}}.
